SEQUENCE LISTING (24 sequences)

SEQ ID NO: 1 - DNA, 22 bp, Artificial
  Primer
aatcccatcaccatcttccagg

SEQ ID NO: 2 - DNA, 22 bp, Artificial
  Primer
agaggcagggatgatgttctgg

SEQ ID NO: 3 - DNA, 24 bp, Artificial
  Primer
tttccagttcagggcagtagtgac

SEQ ID NO: 4 - DNA, 24 bp, Artificial
  Primer
cttccccttcttctccattgtctc

SEQ ID NO: 5 - DNA, 20 bp, Artificial
  Primer
ctggacctcgttgacacctg

SEQ ID NO: 6 - DNA, 20 bp, Artificial
  Primer
gacattctctcgttcaccgc

SEQ ID NO: 7 - DNA, 22 bp, Artificial
  Primer
tcatgatgctgttgtaggtggt

SEQ ID NO: 8 - DNA, 20 bp, Artificial
  Primer
ctgttccagccatccttcat

SEQ ID NO: 9 - DNA, 21 bp, Artificial
  Primer
acatctgcgtccacatcaggg

SEQ ID NO: 10 - DNA, 22 bp, Artificial
  Primer
ccgttcacgagtctcattggtg

SEQ ID NO: 11 - DNA, 21 bp, Artificial
  Primer
atccctgccagaaccaccact

SEQ ID NO: 12 - DNA, 23 bp, Artificial
  Primer
gcgcttctcattctcatggatct

SEQ ID NO: 13 - DNA, 21 bp, Artificial
  Primer
atggcgtcctccctgcttgag

SEQ ID NO: 14 - DNA, 24 bp, Artificial
  Primer
aggggtgtgtcatgtccagagagg

SEQ ID NO: 15 - DNA, 24 bp, Artificial
  Primer
ccttttcaatctgagctcttcttt

SEQ ID NO: 16 - DNA, 20 bp, Artificial
  Primer
ggaagaggagacacggaaca

SEQ ID NO: 17 - DNA, 21 bp, Artificial Sequence
  Primer
atcccatcaccatcttccagg

SEQ ID NO: 18 - DNA, 21 bp, Artificial Sequence
  Primer
gaggcagggatgatgttctgg

SEQ ID NO: 19 - DNA, 22 bp, Artificial Sequence
  Primer
ggcccacaaatctcagatcgtt

SEQ ID NO: 20 - DNA, 20 bp, Artificial Sequence
  Primer
cactggcgctgcaacaagac

SEQ ID NO: 21 - DNA, 20 bp, Artificial Sequence
  Primer
gccaccaaccagccagagaa

SEQ ID NO: 22 - DNA, 25 bp, Artificial Sequence
  Primer
gcgaggtactgagtcttctgaaacc

SEQ ID NO: 23 - DNA, 21 bp, Artificial Sequence
  Primer
acatggaccagcagactggca

SEQ ID NO: 24 - DNA, 20 bp, Artificial Sequence
  Primer
tcactgtcttgccccaggct

(19) United States
(12) Patent Application Publication
(10) Pub. No.: US 2013/0109020 A1
(43) Pub. Date: May 2, 2013

(54) USE OF AN ISOFORM OF HLA-G AS AN OSTEOGENESIS MARKER

(71) Applicants: Frederic Deschaseaux, Tours (FR); Luc Sensebe, Joue Les Tours (FR); Nathalie Rouas-Freiss, Paris (FR); Abderrahim Naji, Carlsbad, CA (US); Edgardo Delfino, Paris (FR)

(72) Inventors: Frederic Deschaseaux, Tours (FR); Luc Sensebe, Joue Les Tours (FR); Nathalie Rouas-Freiss, Paris (FR); Abderrahim Naji, Carlsbad, CA (US); Edgardo Delfino, Paris (FR)

(73) Assignees: Etablissement Francais Du Sang, La Plaine Saint Denis (FR); Commissariat A L'Energie Atomique Et Aux Energies Alternatives, Paris (FR)

(21) Appl. No.: 13/511,488

(22) PCT Filed: Nov. 23, 2010
(86) PCT No.: PCT/IB2010/055380

§ 371 Date: Dec. 28, 2012

(30) Foreign Application Priority Data
Nov. 23, 2009 (FR) 09 05624

(51) Int. Cl. G01N 33/68

(52) U.S. Cl. CPC G01N 33/6893
USPC 435/6.12; 435/7.92; 530/350; 536/23.5

(57) ABSTRACT

The invention relates to the use of at least one isoform of HLA-G as a marker for assessing osteogenesis in mammals.

The present invention relates to a novel osteogenesis marker, and to the use thereof in methods for evaluating osteogenesis in mammals.

Osteogenesis is the process by which bone tissue forms and develops. Bone is formed from:
a bone extracellular matrix (approximately 22% to 25%), which is an organic matrix composed essentially of collagen type I, but also of other proteins such as osteonectin and osteocalcin,
a mineral matrix (approximately 70%) very rich in calcium,
two types of bone cells: osteoblastic cells (osteoblasts, osteocytes and lining cells, which derive from mesenchymal stem cells) and osteoclasts (cells deriving from hematopoietic stem cells), and
water (5% to 8%).

Osteoblasts are cube-shaped or cylinder-shaped mononucleated epithelioid cells present in growing bone tissue. They characteristically express collagen type I, alkaline phosphatase (ALP or ALPL), parathyroid hormone receptor 1 (PTHR1), osteonectin (SPARC), osteocalcin, osterix. transcription factor (OSX) and α-actin (ASMA) (Cohen, 2006).

Osteocytes are differentiated osteoblasts which are highly branched and capable of dividing. They maintain the bone extracellular matrix.

Lining cells are resting cells which have a flattened and elongated shape and which are located at the surface of the bone, in zones which are inactive, i.e. neither undergoing bone formation nor undergoing bone resorption. If they are stimulated, they can differentiate into osteoblasts.

Osteoclasts are multinucleated cells from 20 to 100 μm in diameter. They are responsible for bone resorption.

Having osteogenesis markers is important both for assessing the risks of fracture in individuals suffering from a skeletal degeneration (for example osteoporosis), both for monitoring post-fracture reconstruction of the skeleton, and for monitoring bone tumor development. This risk assessment and this monitoring, if they are early, are essential for setting up effective therapy.

By way of example, 10 to 30% of adults having a fractured tibia exhibit a pseudarthrosis, i.e. an absence of consolidation of the two bone fragments occurring after the fracture. In France, this represents approximately 3000 cases per year. The treatments for pseudarthroses are extremely laborious (autograft, injection of bone marrow, injection of growth factors, etc.) and expensive, and do not enable patients to recover their independence rapidly. The annual cost of a defective consolidation of the tibia can be estimated at between 3 and 30 million euros in France. Early detection of pseudarthrosis would therefore make it possible to treat patients suffering from this condition more effectively.

There are currently few bone formation (osteogenesis) markers on the market. These markers are essentially bone alkaline phosphatase, osteocalcin and procollagen extension propeptides (Srivastava, 2005 and Vesper, 2005):
alkaline phosphatase (ALP or ALPL) is a ubiquitous enzyme. In humans, 6 isoenzymes can be distinguished: hepatic, intestinal, bone, renal, placental and tumor isoenzymes. In adults with normal hepatic function, approximately 60-70% of the serum activity of ALP comes from the liver, 30-40% comes from bone and less than 5% comes from the intestines. The circulating level of bone ALP depends on the activity of the osteoblasts but also on its hepatic elimination; consequently, in the event of a liver condition, there is a possible modification of the level of circulating bone ALP;
osteocalcin (OC) is a 5.7 kDa protein secreted by the osteoblasts. In the bloodstream, osteocalcin is rapidly degraded and eliminated (⅓ of the circulating osteocalcin is represented by the whole protein and ⅔ represented by fragments of various sizes), thereby limiting its clinical application as a bone formation marker;
collagen type I is synthesized by the osteoblasts in the form of procollagen. This precursor contains two propeptides at the N- and C-terminal ends, respectively PINP(N-terminal extension propeptide) and PICP(C-terminal extension propeptide), which are cleaved by proteases during the assembly of procollagen into a triple helix, and released into the circulation. Assaying these propeptides in the serum can therefore make it possible to evaluate the formation of collagen type I. However, this assaying reflects the synthesis of collagen type I not only in the bone but also in other tissues.

Thus, the osteogenesis markers currently used are not entirely satisfactory. This therefore results in a need to identify new osteogenesis markers.

The major histocompatibility complex (MHC) antigens are divided up into several classes: class I antigens (HLA-A, HLA-B and HLA-C) which have 3 globular domains (α1, α2 and α3) and the α3 domain of which is associated with β2-microglobulin, class II antigens (HLA-DP, HLA-DQ and HLA-DR) and class III antigens (complement). The class I antigens comprise, in addition to the abovementioned antigens, other antigens, termed unconventional class I antigens (class Ib) and in particular the HLA-E, HLA-F and HLA-G antigens.

The nucleotide sequence of the HLA-G gene (HLA-6.0 gene) has been described by Geraghty et al. (1987): it comprises 4396 base pairs and has an intron/exon organization homologous to that of the HLA-A, -B and -C genes. This gene comprises 8 exons, 7 introns and an untranslated 3′ end. The HLA-G gene differs from the other MHC Class I genes in that the in-frame translation stop codon is located at the second codon of exon 6; consequently, the cytoplasmic region of the protein encoded by this HLA-6.0 gene is shorter than that of the cytoplasmic regions of the HLA-A, -B and -C proteins.

Ishitani and Geraghty (1992) have shown that the primary transcript of the HLA-G gene can be spliced in several ways and produces at least 3 distinct mature mRNAs: the primary HLA-G transcript provides a full-length copy (G1) of 1200 bp, a fragment of 900 bp (G2) and a fragment of 600 bp (G3). The G1 transcript does not comprise exon 7 and corresponds to the sequence described by Ellis et al. (1990), i.e. it encodes a protein which comprises a signal sequence, three extra-cellular globular domains (α1, α2 and α3), a trans-membrane domain and an intracytoplasmic domain. The G2 mRNA does not comprise exon 3 (encoding the α2 domain), and encodes an isoform in which the α1 and α3 domains are directly joined. The G3 mRNA contains neither exon 3 nor exon 4 (encoding the α3 domain); this transcript therefore encodes an isoform in which the α1 domain and the transmembrane domain are directly joined. The splicing which prevails in order to obtain the HLA-G2 antigen leads to the joining of an adenine (A) (originating from the α1 coding domain), with an adenine-cytosine (AC) sequence (derived from the α3 coding domain), which leads to the creation of an AAC (asparagine) codon in place of the GAC (aspartic acid) codon, present in the 5′ position of the sequence encoding the α3 domain in HLA-G1. The splicing generated in order to obtain HLA-G3 does not lead to the formation of a new codon in the splicing region.

Some of the inventors have shown the existence of other spliced forms of HLA-G mRNA: the HLA-G4 transcript, which does not include exon 4; the HLA-G5 transcript, which includes intron 4, between exons 4 and 5, thus causing a modification of the reading frame, during the translation of this transcript, and in particular the appearance of a stop codon after amino acid 21 of intron 4; the HLA-G6 transcript, which has intron 4, but which has lost exon 3; and the HLA-G7 transcript which includes intron 2, thus causing a modification of the reading frame, during the translation of this transcript, and the appearance of a stop codon after amino acid 2 of intron 2 (Kirszenbaum et al., 1994 and 1995; Moreau et al., 1995; European application EP 0 677 582).

There are therefore at least 7 different HLA-G mRNAs which encode 7 isoforms of HLA-G, 4 of which are membrane isoforms (HLA-G1, -G2, -G3 and -G4) and 3 of which are soluble isoforms (HLA-G5, -G6 and -G7), which do not comprise a transmembrane domain) (for review see Carosella et al., 2008a).

The nucleotide sequence of the HLA-G gene (HLA-6.0 gene) and its exon/intron organization, and also the amino acid sequences of the various isoforms of HLA-G are well known to those skilled in the art. They have in particular been described by Geraghty et al. (1987) and by Carosella et al. (2008a) mentioned above.

HLA-G protein expression is normally restricted to trophoblasts (at the maternal-fetal interface), to the thymus, to the cornea, to endothelial and erythroblast precursors and to mesenchymal stem cells (Carosella et al., 2008a). However, HLA-G mNRAs are detected in virtually all cells of the body at a basal level which can be amplified and the translation of which into protein is induced under the effect of DNA-demethylating agents, of cytokines such as interferons (IFN), of stress factors or of hypoxia (Carosella et al., 2008b). Thus, under particular conditions, such as the transplantation of a tissue, the development of certain tumors or an inflammatory response, the HLA-G protein may be expressed in tissues which do not express it under normal conditions.

In the blood, both the soluble isoforms and the membrane isoforms detached from the membrane, such as HLA-G1 (which is also known as HLA-G1s, for “HLA-G1 shedding”) are found.

Several biological properties of HLA-G have been identified: inhibition of NK-cell-mediated and CTL-mediated cytolysis, inhibition of the alloproliferative T response, induction of apoptosis in CD8⁺NK cells and T cells, and an antiproliferative action on B cells of the immune system (Carosella et al., 2008a and 2008b).

Some of the inventors have recently shown that mesenchymal stem cells (MSCs) in culture secrete HLA-G5, thus exerting an immunosuppressive activity with respect to the T and NK (Natural Killer) immune response (Selmani et al., 2008).

Mesenchymal stem cells derived from adult bone marrow are multipotent cells that are precursors of osteoblasts, of chondroblasts and of adipocytes (Friedenstein et al., 1976; Pittenger et al., 1999).

In the context of these studies, the inventors have investigated whether the osteoblasts, chondroblasts and adipocytes obtained in culture from MSCs can, themselves also, express HLA-G. Surprisingly, the inventors have shown, via the ELISA method, that only the osteoblasts express the soluble HLA-G forms. They have also shown, via the RT-PCR technique, that the osteoblasts express HLA-G1, -G2, -G3, -G4 and -G5 mRNAs. In addition, the inventors have observed, in humans, in vivo, that HLA-G is expressed by normal or pathological (in particular tumor) osteoblasts only during bone formation (osteogenesis).

On the basis of these results showing HLA-G expression by osteoblasts during bone formation, the evaluation of osteogenesis in an individual, whether during a post-fracture bone reconstruction or in the context of monitoring the progression of a bone tumor, can therefore be carried out by assaying or determining the expression, by the osteoblasts, of at least one isoform of HLA-G: an increase in the concentration or in the expression of at least one isoform of HLA-G being an indication of osteogenesis.

Consequently, the subject of the present invention is an in vitro method for monitoring bone reconstruction in a subject, in whom a bone fracture has been diagnosed, which method comprises the following steps:
a) measuring the concentration of at least one isoform of HLA-G in a sample of biological fluid from said subject,
b) comparing the concentration of the isoform(s) of HLA-G measured in step a) with a reference concentration of this or these isoform(s) of HLA-G in said biological fluid in healthy subjects,
wherein a concentration of at least one isoform of HLA-G, in said sample of biological fluid from said subject, which is higher than said reference concentration indicates bone reconstruction (osteogenesis) in said subject.

The term “bone fracture” is intended to mean a break in the continuity of a bone. This may in particular be a bone fissure (fracture without displacement of the bone) or a comminuted fracture (comprising several bone fragments; multisplintered fracture). A bone fracture can be diagnosed, for example, by radiography, scintigraphy or tomodensitometry.

For the purpose of the present invention, the term “subject” is intended to mean a mammal, preferably a human being.

For the purpose of the present invention, the term “healthy subjects” is intended to mean subjects who do not have a bone lesion, i.e. a bone fracture.

For the purpose of the present invention, the term “biological fluid” is intended to mean blood and derivatives thereof (such as plasma and serum) and also synovial fluid, preferably blood.

For the purpose of the present invention, the term “isoform of HLA-G” is intended to mean an isoform of HLA-G chosen from the membrane isoforms (HLA-G1, -G2, -G3 and -G4) and soluble isoforms (HLA-G5, -G6 and -G7) of HLA-G.

Of course, when the concentration of a membrane isoform of HLA-G is measured in a biological fluid, this means that this isoform is detached from the cell membrane (by proteolysis, for example) and is contained in said biological fluid.

According to one preferred embodiment of said method for monitoring bone reconstruction, the concentration of at least one isoform of HLA-G chosen from HLA-G1, HLA-G5, HLA-G6 and HLA-G7 is measured.

According to another preferred embodiment of said method for monitoring bone reconstruction, the concentration of two different isoforms of HLA-G, preferably HLA-G1 and HLA-G5, or HLA-G5 and HLA-G6, is measured.

According to another preferred embodiment of said method for monitoring bone reconstruction, the plasma or serum concentration of at least one isoform of HLA-G as defined above, preferably chosen from HLA-G1, HLA-G5, HLA-G6 and HLA-G7, or more preferably of two different isoforms of HLA-G, such as HLA-G1 and HLA-G5, and HLA-G5 and HLA-G6, is measured using a blood sample.

The measurement of the plasma concentration of an isoform of HLA-G using a blood sample is well known to those skilled in the art. It can be carried out by implementing a suitable immunological method (e.g. ELISA, RIA, immunofluorescence, immunohistochemistry) by means of at least one antibody specific for said isoform of HLA-G.

For the purpose of the present invention, the term “antibody” is intended to mean a polyclonal or monoclonal antibody which is human or nonhuman, for example murine, humanized, chimeric; recombinant or synthetic; or an antibody fragment (for example the Fab′2 or Fab fragments) comprising a domain of the initial antibody which recognizes the target antigen of said initial antibody.

Many monoclonal or polyclonal antibodies specific for one or more isoforms of HLA-G are known to those skilled in the art. By way of example of commercially available anti-human HLA-G monoclonal antibodies, mention may be made of the anti-HLA-G antibody (which recognizes all isoforms of HLA-G) obtained from the 4H84 clone (McMaster et al., 1998), the anti-HLA-G1, -G2, -G5 and G6 antibody (i.e. which recognizes the HLA-G1; -G2, -G5 and G6 isoforms) obtained from the MEM-G/4 clone (also called MEM-G/04; Menier et al., 2003), the anti-HLA-G1 and anti-HLA-G5 antibodies (i.e. which recognize the HLA-G1 and -G5 isoforms) obtained from the MEM-G/9 clone (also called MEM-G/09; Menier et al., 2003) or the 87G clone (Rebmann et al., 1999; Hackmon et al., 2004), and the anti-HLA-G5 and anti-HLA-G6 antibody (i.e. which recognizes the HLA-G5 and -G6 isoforms) obtained from the 5A6G7 clone (Le Rond et al., 2004).

The reference concentration of an isoform of HLA-G in a biological fluid as defined above depends not only on the given isoform of HLA-G, but also on the method used to measure the concentration.

The reference plasma concentration of the HLA-G1 (HLA-G1s) and HLA-G5 isoforms in healthy individuals, i.e. individuals with no bone fracture, measured by ELISA using the monoclonal antibody obtained from the MEM-G/9 clone, is less than 20 ng/ml (Ugurel et al., 2001; Le Rond et al., 2006; Naji et al., 2007).

Thus, according to one particular arrangement of this embodiment, a plasma concentration of the HLA-G1 (HLA-G1s) and HLA-G5 isoforms of greater than 20 ng/ml, preferably greater than 50 ng/ml, more preferably greater than 100 ng/ml, measured by means of an appropriate immunological method, preferably via ELISA, using an antibody which is both anti-HLA-G1 and anti-HLA-G5 (for example, the monoclonal antibody obtained from the MEM-G/9 clone), using a blood sample from said human subject in whom a bone fracture has been diagnosed, indicates bone reconstruction in said subject.

The reference plasma concentration of the HLA-G5 and HLA-G6 isoforms in said healthy individuals, measured by ELISA using the monoclonal antibody obtained from the 5A6G7 clone, is less than 10 ng/ml (Le Rond et al., 2006; Naji et al., 2007).

Thus, according to another particular arrangement of this embodiment, a plasma concentration of the HLA-G5 and HLA-G6 isoforms of greater than 10 ng/ml, preferably greater than 50 ng/ml, more preferably greater than 100 ng/ml, measured by means of an appropriate immunological method, preferably by ELISA, using an antibody which is both anti-HLA-G5 and anti-HLA-G6 (for example, the monoclonal antibody obtained from the 5A6G7 clone), using a blood sample from said human subject in whom a bone fracture has been diagnosed, indicates bone reconstruction in said subject.

The reference serum concentration of the HLA-G1 (HLA-G1s) and HLA-G5 isoforms in said healthy individuals, measured by ELISA using the monoclonal antibody obtained from the MEM-G/9 clone, is approximately 20 ng/ml (Rouas-Freiss et al., 2005).

Thus, according to another particular arrangement of this embodiment, a plasma concentration of the HLA-G1 (HLA-G1s) and HLA-G5 isoforms of greater than 25 ng/ml, preferably greater than 50 ng/ml, more preferably greater than 100 ng/ml, measured by means of an appropriate immunological method, preferably via ELISA, using an antibody which is both anti-HLA-G1 and anti-HLA-G5 (for example, the monoclonal antibody obtained from the MEM-G/9 clone), using a blood sample from said human subject in whom a bone fracture has been diagnosed, indicates bone reconstruction in said subject.

The present invention also relates to an in vitro method for monitoring the change (progression or remission) in a bone tumor in a subject (in whom a bone tumor has been diagnosed), using biological samples from said subject obtained at a time t0 and at a time t1, which method comprises a step of determining the concentration or of quantitatively determining the expression of at least one isoform of HLA-G in said biological samples:
wherein an increase in the concentration or in the expression level of at least one isoform of HLA-G between the times t0 and t1 indicates a progression of said bone tumor in said subject,
wherein a decrease in the concentration or in the expression level of at least one isoform of HLA-G between the times t0 and t1 indicates a remission (or regression) of said bone tumor in said subject.

For the purpose of the present invention, the term “biological sample” is intended to mean a sample of biological fluid as defined above or a biological sample comprising osteoblasts which has been obtained by bone biopsy of the tumor.

The biopsy may be a sample taken from any part of the skeleton, such as the spine, the pelvis, the femur, the tibia, the humerus, the shoulder blade and the skull.

The bone tumor is chosen from the group consisting of osteosarcoma, osteoblastoma, Ewing's sarcoma and giant-cell tumors.

According to one preferred embodiment of this method for in vitro evaluation of a bone tumor, the plasma or serum concentration of at least one isoform of HLA-G as defined above is determined, for example by means of an appropriate immunological method as defined above, using a blood sample from said human subject.

According to one advantageous arrangement of this embodiment, the concentration of two different isoforms of HLA-G, preferably HLA-G1 and HLA-G5, or HLA-G5 and HLA-G6, is determined.

According to another preferred embodiment of this method for in vitro evaluation of a bone tumor, the expression, by the osteoblasts, of at least one isoform of HLA-G as defined above is quantitatively determined using biological samples comprising osteoblasts, which samples were obtained by bone biopsy of said tumor.

According to one advantageous arrangement of this embodiment, the expression of at least one isoform of HLA-G by the osteoblasts is determined in vitro by means of an immunological method as defined above, preferably an appropriate immunohistochemical method, by means of at least one antibody specific for said isoform of HLA-G as defined above.

According to another advantageous arrangement of this embodiment, the expression of at least one isoform of HLA-G by the osteoblasts is determined in vitro by detecting the mRNAs encoding at least one isoform of HLA-G. The detection of the mRNAs can be carried out by hybridization, by means of nucleotide probes specific for said mRNAs (attached, for example, to a biochip), or by amplification (for example by RT-PCR), by means of nucleotide primers specific for said mRNAs. By way of example of primers suitable for amplifying HLA-G mRNAs, mention may be made of the pair of primers consisting of the nucleotide sequences SEQ ID No.s 15 and 16, and those described by Le Discorde et al., 2005.

The subject of the present invention is also a method of in vitro screening for an agent which modulates (increases or decreases) osteogenesis, which method comprises the following steps:
a) quantitatively determining the expression of at least one isoform of HLA-G by osteoblasts;
b) bringing said osteoblasts into contact with a test agent; then
c) quantitatively determining the expression of said at least one isoform of HLA-G by said osteoblasts,
wherein a difference in the level of expression, by said osteoblasts, of at least one isoform of HLA-G, between steps a) and c), indicates that said agent modulates osteogenesis.

An increase in the level of expression, by said osteoblasts, of at least one isoform of HLA-G, between steps a) and c) above, indicates that said agent induces an osteogenesis process.

A decrease in the level of expression, by said osteo-blasts, of at least one isoform of HLA-G, between steps a) and c) above, indicates that said agent suppresses an osteogenesis process.

The quantitative determination of the expression of at least one isoform of HLA-G by the osteoblasts can be carried out by means of a method as described above, i.e. by means of an immunological method or by detection of the mRNAs encoding the isoforms of HLA-G.

According to one preferred embodiment of said screening method, the osteoblasts are of human origin.

According to another preferred embodiment of said screening method, the expression of HLA-G1 and HLA-G5, or of HLA-G5 and HLA-G6, is determined.

The subject of the present invention is also an isoform of HLA-G or a nucleic acid molecule (for example an mRNA) encoding an isoform of HLA-G, isolated from a subject, preferably a human being, for use as a marker, preferably as a blood marker, for osteogenesis in said subject.

According to one preferred embodiment of the invention, said isoform of HLA-G is chosen from HLA-G1, HLA-G5 and HLA-G6.

In addition to the above arrangements, the invention also comprises other arrangements, which will emerge from the description that follows, which refer to exemplary embodiments of the method which is the subject of the present invention and also to the appended drawings, in which:

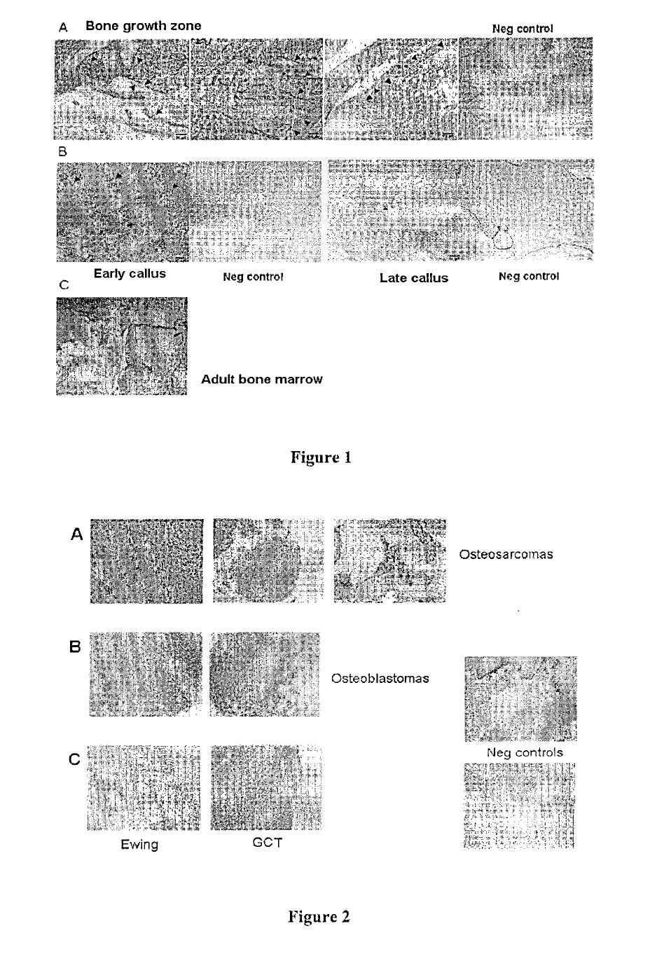

FIG. 1 shows the expression of HLA-G by the osteoblasts of the bone growth zones in a newborn baby (A) and of post-fracture calluses in an adult (B). Thin sections of supernumerary digits and of early and late calluses were incubated with an anti-HLA-G5 and anti-HLA-G6 antibody (clone 5A6G7) and then with a peroxidase-conjugated anti-mouse secondary antibody. The slides were then observed under a photon microscope. In the bone growth zones, the osteoblasts located along the bone trabeculae and the periosteum are labeled with the anti-HLA-G5/-G6 antibody (see black arrows). In the bone calluses, the condensed osteoblasts at newly formed bone trabeculae are also labeled with the anti-HLA-G5/-G6 antibody. (C): no HLA-G+ cell could be observed in a nonpathological adult bone marrow;

FIG. 2 shows the detection of HLA-G in bone tumors. Thin sections of biopsies of bone tumors originating from osteosarcomas (A), from osteoblastomas (B), from Ewing's sarcomas and from giant-cell tumors (C) were incubated with an anti-HLA-G5/-G6 antibody (clone 5A6G7). The tumor osteoblasts of osteosarcomas and of osteoblastomas are labeled (A and B), whereas only the normal osteoblasts of the tumor micro-environment of the Ewing's sarcomas or of giant-cell tumors are positive for HLA-G5/-G6 expression (C);

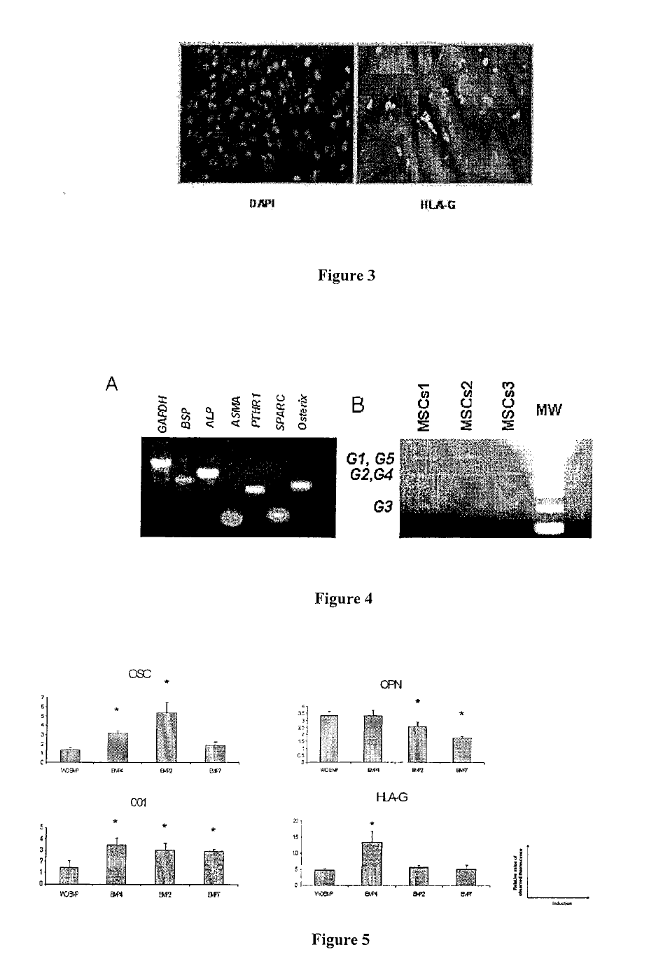

FIG. 3 shows the detection of HLA-G in the SaOs2 line by in situ immunofluorescence. The cells of the SaOs2 osteosarcoma line were cultured in a culture chamber containing wells, then fixed and incubated with an anti-HLA-G1 and anti-HLA-G5 antibody (clone 87G) conjugated to FITC. The cell nuclei were labeled with DAPI. Magnification ×400;

FIG. 4 shows the expression of HLA-G mRNA by osteoblasts obtained from MSCs. The osteoblasts were lysed and the mRNA recovered. After reverse trans-cription, the cDNAs were amplified by PCR. (A): the transcripts (BSP, ALP, PTHR1, SPARC, Osterix and ASMA mRNAs) characteristic of osteoblasts were sought on the cells cultured in the osteogenic medium. GAPDH (glyceraldehyde-3-phosphate dehydrogenase; constitutive gene) mRNA was used as a control. (B): the expression of the various forms of HLA-G (G1, G2, G3, G4 and G5) in 3 different osteoblast cultures obtained from MSCs (MSC1, MSC2, MSC3) was sought;

FIG. 5 shows the expression of HLA-G by osteoblasts obtained from MSCs, via in situ immuno-fluorescence. The MSCs were cultured in the culture chamber containing wells and at confluence, and induced so as to differentiate into osteoblasts. The inducers used were: BMP-2, BMP-4 and BMP-7. MSCs cultured in a non-inducing medium (expansion medium) were used as negative control (WOBMP). After 10 days of culture, the osteoblasts were fixed and permeabilized before being incubated with the anti-collagen 1 (CO1), anti-osteopontin (OPN), anti-osteocalcin (OSC) and anti-HLA-G1/-G5 (clone 87G) antibodies. The cell nuclei were labeled by adding DAPI to the reaction medium. The fluorescence was then detected under an epifluorescence microscope. The fluorescences emitted by the presence of the antibodies were related back to that emitted by the DAPI so as to take into account a level of expression of the molecules sought relative to the number of cells present in the wells (“relative value of observed fluorescence”);

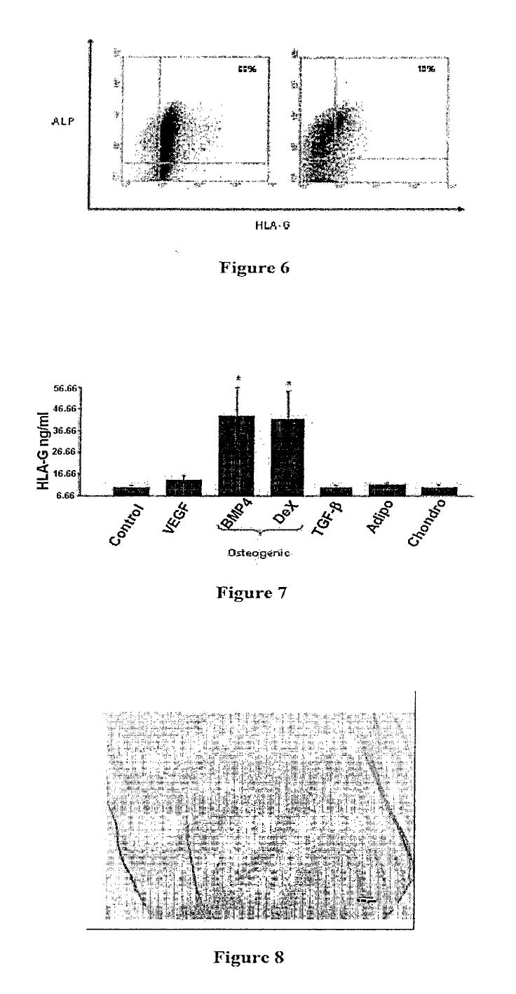

FIG. 6 shows the expression of HLA-G by flow cytometry during osteogenesis. The MSCs were cultured in an osteogenic medium and the joint expressions of HLA-G5/-G6 and of alkaline phosphatase (ALP) were monitored by flow cytometry at the 2nd (A) and 4th (B) week of culture. The number of HLA-G+ cells decreases from 66% (week 2) to 10% (week 4). The Figure is representative of 4 independent experiments; the values given are the means observed over all the experiments;

FIG. 7 shows the assaying of soluble HLA-G in the culture supernatants of osteoblasts obtained from MSCs. The MSCs were cultured in media inducing osteogenic differentiation (BPM4, dexamethasone), chondrogenic differentiation (Chondro, TGF-β), adipogenic differentiation (Adipo) and angiogenic differentiation (VEGF) or in the absence of any induction (control). After 4 days of incubation, aliquots of the supernatants were taken and the HLA-G5 and -G6 isoforms therein were assayed by ELISA. The results are reported in ng/ml;

FIG. 8 shows the search for the expression of HLA-G in chondrocytes after micromass culture. The MSCs were cultured under micromass conditions in a chondrogenic medium. After 4 weeks of culture, the micromasses were recovered, fixed and paraffin-embedded. The rehydrated thin sections were incubated either with an anti-HLA-G antibody (clone 5A6G7) or with a control antibody (isotype control). After washing, the sections were stained with hematoxylin/eosin and then observed under a photon microscope;

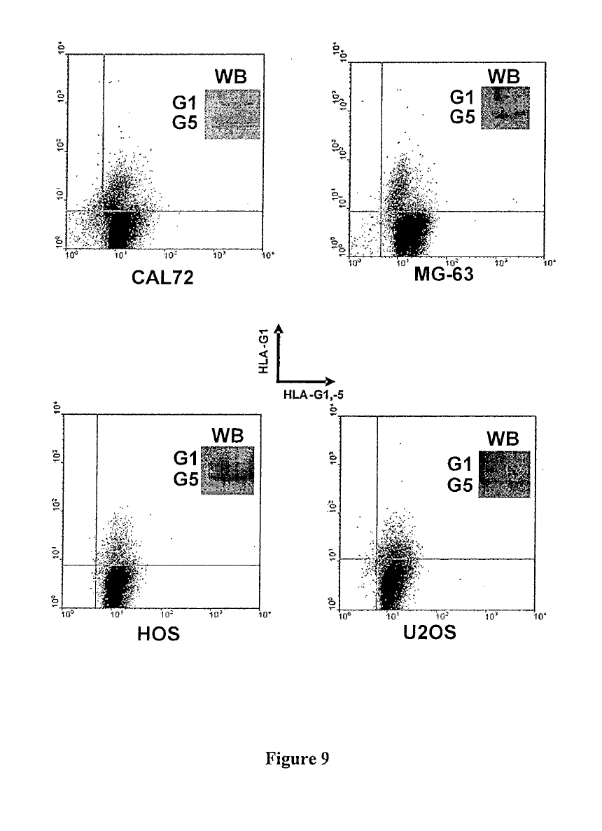

FIG. 9 shows the expression of HLA-G1 and of HLA-G5 by the human osteoblast lines CAL72, MG-63, HOS and U2OS derived from osteosarcomas (OS). WB=Western Blot;

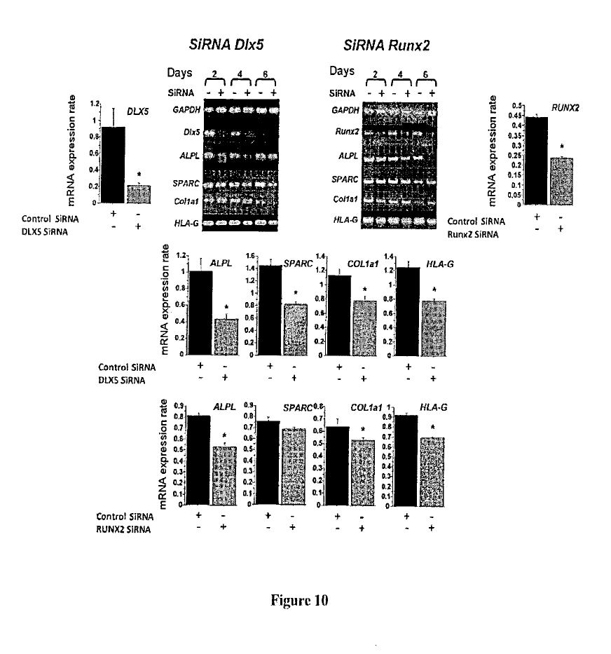

FIG. 10 shows the effect of inhibition of the DLX5 or RUNX2 genes on the expression of GAPDH, ALPL, SPARC, COL1α1, HLA-G1 and HLA-G5 in human bone marrow mesenchymal stem cells induced so as to differentiate into osteoblasts. The analysis was carried out by RT-PCR;

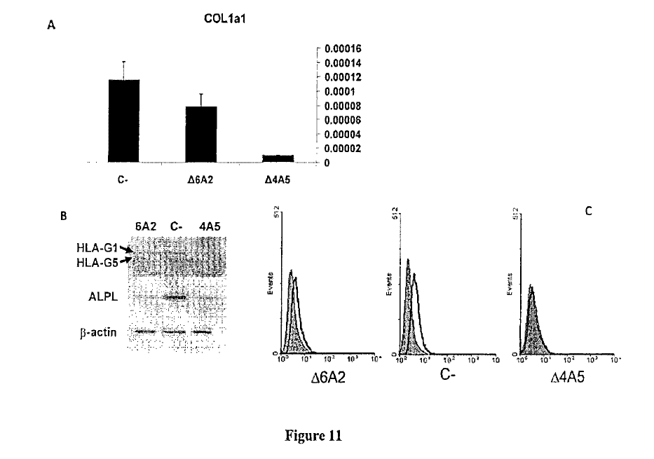

FIG. 11 shows the expression of HLA-G1 and -G5, COL1α1 and ALPL by the osteoblast lines of human origin Δ4A5 and Δ6A2, overexpressing RUNX2. A: analysis by qPCR. B: analysis by Western blot; the HLA-G1 and HLA-G5 isoforms were detected, the β isoform of actin was used as an internal positive control. C: analysis by flow cytometry; the expression of HLA-G1 and -G5 is shown by the thick-line histogram, while the negativity threshold is given by the histogram of the isotype control (in gray).

### EXAMPLE 1

### Demonstration of the Expression of HLA-G by Osteoblasts

### 1) Materials and Methods

### Cells and Cell Cultures:

The adult human bone marrow (BM) samples were collected from healthy volunteers undergoing orthopedic surgery. These specimens were taken according to the recommendations of the ethics committee of the Trousseau CHU [University teaching hospital center] in Tours, France. The mononuclear cells (MNCs) were seeded in alpha-MEM medium (MEM) supplemented with 10% fetal calf serum (FCS) (Perbio Hyclone; Logan) and 1% penicillin/streptomycin (InVitrogen Ltd; Paisley, UK). On the 14th day, at 80% confluence, the mesenchymal stem cells (MSCs) were duplicated and amplified. All the experiments were carried out with at least 3 different donors.

The SaOs2 osteosarcoma line (ECECC, Salisbury, UK) was cultured under the same conditions as for the expansion of the MSCs.

### Osteoblastic and Chondroblastic Induction:

The MSCs were induced to differentiate into osteoblasts and chondrocytes according to the protocols described by Pittenger et al., 1999.

Briefly, in order to induce osteogenesis, the medium is composed of DMEM 4.5 g/l glucose, 3 mM NaH₂PO₄ (InVitrogen), 25 mg/l ascorbic acid (Sigma) and 10⁻⁷ M dexamethasone (InVitrogen). This medium enables the cells to differentiate into osteoblasts after 14 days of culture.

In order to induce chondrogenesis, the cells were centrifuged and micromass-cultured, i.e. cultured in a pellet directly without resuspension. The differentiation medium used is composed of DMEM 3M glucose (InVitrogen), 2 mM sodium pyruvate (Sigma, Saint Quentin Fallavier, France), 0.17 mM ascorbic acid 2-phosphate (Sigma), 10⁻⁷ M dexamethasone (InVitrogen), 0.35 mM proline (Sigma) and a 1× insulin-transferrin-selenium (ITS) supplement (Sigma). Transforming Growth Factor beta type 1 (TGFβ1) (AbCys) was added at a concentration of 10 ng/ml during each renewal of medium. This medium allows chondrogenic differentiation after 21 days of culture.

### Analyses by Flow Cytometry:

For the membrane and intracytoplasmic detection of an antigen, 200,000 cells were labeled with a specific monoclonal antibody coupled to a fluorochrome. The membrane labeling was obtained after 30 minutes of incubation at 4° C. in the dark. The cells were then rinsed with phosphate buffered saline (PBS), and then fixed with CellFIX® (Becton Dickinson, Erembodegem, Belgium). For the intracellular labeling, the cells were fixed and permeabilized using the CytoFix/CytoPerm kit. The cells were then run through a flow cytometer (FACS Calibur®, Becton Dickinson), equipped with an Argon laser, emitting a wavelength of 488 nm. The data were analyzed using the CellQuest 3.1® software (Becton Dickinson); the results are expressed as ratio of mean fluorescence intensity (RMFI) of the signal detected over that of the background noise. The following monoclonal antibodies (mAbs) were used: anti-HLA-G1/-G5 mAb (clone 87G) conjugated to Alexa 488 (Exbio, Vestec, Czech Republic), anti-HLA-G5/-G6 mAb (clone 5A6G7, Exbio) and the anti-alkaline phosphatase (ALP) mAb conjugated to phycoerythrin (PE).

### Analyses by ELISA (Enzyme Linked Immunosorbent Assay):

The concentrations of soluble HLA-G contained in the filtered supernatants of MSC cultures after osteoblastic, chondrocytic, adipocytic and vascular inductions were measured. The culture supernatant of cells of the M8-HLA-G5 line was used as a positive control (Le Rond et al., 2006). The ELISA method used comprises the use of the anti-HLA-G5/-G6 antibody (clone 5A6G7, Exbio) as capture antibody and the pan-HLA class I W6/32 as antibody for detecting HLA class Ia molecules (Betts et al., 2003) (Rebmann et al., 2005).

### Analyses by In Situ Immunofluorescence:

The cells selected from 3 fresh tumors were seeded into culture chambers containing wells with a surface area of 1 cm² (Labteks®, Nunc International, Rochester, N.Y., USA) at 10 000 cells/well. After 48 hours of culture in proliferation medium, they were fixed for 10 minutes with 3.7% formaldehyde (Sigma) or with pure methanol (InVitrogen). A permeabilization step with a solution of PBS, 0.5% FCS and 0.2% Tween 20 (BioRad, Hercules, Calif., USA), for 30 minutes at ambient temperature, was necessary in order to identify the intracellular proteins. The cells were then incubated successively with the monoclonal primary antibodies, for 1 hour at 4° C., and the secondary antibodies, which recognize primary antibodies, conjugated to a fluorochrome of Alexa 488 or 594 type (InVitrogen). After rinsing, a mounting medium containing 4,6-di-amidino-2-phenylindole (DAPI) (Vector Cliniscience, Montrouge, France) was added, in order to visualize the cell nuclei. Wells without antibody solution served as a negative control. The slides were read under an epifluorescence microscope (Leica®, Solms, Germany) equipped with a camera (DMX 1200, Nikon Europe, Badhoevedorp, the Netherlands). The image processing was carried out using the Lucia software. The antibodies used were the following: anti-HLA-G1/-G5 monoclonal antibody (clone 87G, Exbio), anti-osteocalcin (Santa Cruz; Tebu, Le Peray en Yvelines, France), anti-osteopontin (RnD) and anti-collagen 1a1 (Santa Cruz) polyclonal antibodies.

### Immunohistochemical Analyses:

The biopsy samples were fixed with 10% formaldehyde (Sigma). They were then dehydrated using successive baths of 75%, 90% and 100% ethanol (Merck and Carbo Erba, Saint Herblain, France). Finally, they were placed in a xylene bath (InVitrogen) for 30 minutes at 4° C., before being paraffin-embedded (InVitrogen) in order to cut sections on a microtome. The sections were then stained with hematoxylin and eosin. The immunolabelings for detecting the antigen were carried out using an anti-HLA-G5/-G6 monoclonal antibody (clone 5A6G7) and a peroxidase-conjugated anti-mouse polyclonal antibody.

### Analyses by RT-PCR:

After cell lysis with Trizol® (InVitrogen), 200 μl of chloroform (Sigma) were added in order to separate the cell and protein debris and also the deoxyribonucleic acid (DNA) and ribonucleic acid (RNA) contained in the supernatant. The RNA then recovered was precipitated with 500 μl of isopropanolol (Sigma), then rinsed with 1 ml of ethanol at 75° C. (Merck and Carbo Erba) and dried in the open air for 30 minutes, before being again dissolved in 50 ml of DNAse- and RNAse-free DEPC water.

The Takara PrimeScript™ 1st strand cDNA synthesis kit (Takara Bio Inc.) was used to perform the complementary DNA (cDNA) synthesis by reverse transcription from the RNAs.

A solution of 1 μg of RNA was added to a first reaction mixture composed of 1 μl of oligo dT primer, 1 μl of dNTP mixture, and RNAse-free water up to a total mixture volume of 10 μl. After a denaturation phase of 5 min at 65° C., a new reaction mixture was added. It was composed of 4 μl of buffer, 1 μl of Prime Script™ reverse transcriptase enzyme, 0.5 μl of RNase enzyme inhibitor and 4.5 μl of DEPC water.

The final mixture of 20 μl was then placed in a thermocycler (Applied Biosystems, Foster City, Calif., USA). The conditions used were: primer/RNA hybridization for 10 min at 30° C.; cDNA synthesis for 60 min at 42° C.; and enzyme inactivation for 5 min at 95° C.

The TaKaRa Ex Taq™ kit (Takara Bio Inc.) was used to carry out the amplification of a desired DNA sequence.

A solution of cDNA at 50 ng/μl was added to a reaction mixture composed of 2.5 μl of buffer, 2 μl of dNTP mixture, 2 μl of primer for the DNA studied, 0.125 μl of Taq polymerase enzyme, and DEPC water up to a total mixture volume of 25 μl. The RT-PCRs were carried out according to the following program: 5 min at 94° C.; 35 cycles (45 sec at 94° C., 45 sec at 55° C., 1 min at 72° C.); 7 min at 70° C.

The primers for amplifying the various genes characteristic of osteogenic differentiation, of immaturity and of cell tumorogenicity are given in table I hereinafter.

TABLE I

| | Primers: |
|---|---|
| Genes | sense antisense |
| GAPDH | AATCCCATCACCATCTTCCAGG |
| | (SEQ ID NO: 1) |
| | AGAGGCAGGGATGATGTTCTGG |
| | (SEQ ID NO: 2) |
| BSP | TTTCCAGTTCAGGGCAGTAGTGAC |
| | (SEQ ID NO: 3) |
| | CTTCCCCTTCTTCTCCATTGTCTC |
| | (SEQ ID NO: 4) |
| PA | CTGGACCTCGTTGACACCTG |
| | (SEQ ID NO: 5) |
| | GACATTCTCTCGTTCACCGC |
| | (SEQ ID NO: 6) |
| α-SM actin | TCATGATGCTGTTGTAGGTGGT |
| | (SEQ ID NO: 7) |
| | CTGTTCCAGCCATCCTTCAT |
| | (SEQ ID NO: 8) |
| PTHR1 | ACATCTGCGTCCACATCAGGG |
| | (SEQ ID NO: 9) |
| | CCGTTCACGAGTCTCATTGGTG |
| | (SEQ ID NO: 10) |
| SPARC | ATCCCTGCCAGAACCACCACT |
| | (SEQ ID NO: 11) |
| | GCGCTTCTCATTCTCATGGATCT |
| | (SEQ ID NO: 12) |
| Osterix | ATGGCGTCCTCCCTGCTTGAG |
| | (SEQ ID NO: 13) |
| | AGGGGTGTGTCATGTCCAGAGAGG |
| | (SEQ ID NO: 14) |
| HLA-G | CCTTTTCAATCTGAGCTCTTCTTT |
| | (SEQ ID NO: 15) |
| | GGAAGAGGAGACACGGAACA |
| | (SEQ ID NO: 16) |

### 2) Results

### A) In Vivo Expression of HLA-G by Osteoblasts During Bone Formation

In newborn babies, the osteoblasts lining the bone trabeculae of spongy bone (or bone marrow) express HLA-G5 and -G6, as attested to by the osteoblast staining obtained with the anti-HLA-G5/-G6 antibody (clone 5A6G7) (see FIG. 1A). Conversely, proliferative and hypertrophic chondrocytes do not express HLA-G5/-G6. Moreover, it should be noted that certain perivascular cells of vessels close to HLA-G+ osteoblasts (labeled with the anti-HLA-G5/-G6 antibody) also express this protein.

In healthy adults, HLA-G5 and -G6 were detected during active osteogenesis such as at the time of post-fracture bone reconstruction. At the time of post-fracture bone reconstruction, during the early phase of bone consolidation, the mesenchymal cells condense so as to generate osteoblasts capable of forming bone. In this early post-fracture phase, the osteoblasts weakly express HLA-G5 and -G6, whereas, in the later phases of bone reconstruction, the osteoblasts responsible for bone neosynthesis within the bone callus are very strongly labeled with the anti-HLA-G5/-G6 antibody (see FIG. 1B). Moreover, HLA-G5 and -G6 were not detected in the nonpathological normal bone marrow of a subject (see FIG. 1C).

These results show that osteoblasts are capable of expressing HLA-G. However, no HLA-G+ cell could be observed in nonpathological adult bone marrow. It therefore emerges from these results that, in vivo, in humans, HLA-G is expressed by osteoblasts during bone formation (osteogenesis). Moreover, perivascular cells closely linked to the osteoblasts can themselves also express HLA-G.

### B) HLA-G is Expressed by Normal and Pathological Osteoblasts

The HLA-G5 and -G6 expression profile on the basis of sections of human pathological bone marrow, originating from osteosarcomas, osteoblastomas, Ewing's sarcomas or giant-cell tumors (GCTs), was studied.

The results represented in FIG. 2 show that, irrespective of the pathological bone marrow studied, the osteoblasts are labeled with the anti-HLA-G5/-G6 antibody (HLA-G+). However, the expression of HLA-G5 and -G6 is more or less sizable depending on the pathological condition.

More specifically, the abnormal osteoblasts originating from osteosarcomas and from osteoblastomas, and also the osteoblasts that are normal but peripheral to the tumor are HLA-G+ (see FIGS. 2A and 2B).

On the sections of bone marrow originating from giant-cell tumors (GCTs) or from Ewing's sarcomas, HLA-G5 and -G6 were detected only in the osteoblasts of the tumor environment (see FIG. 2C).

The expression of HLA-G was confirmed by studying the SaOs2 tumor line derived from an osteoblastic osteosarcoma. The results, represented in FIG. 3, show that the cells are labeled with the 87G antibody which recognizes HLA-G5 (soluble form of HLA-G) and HLA-G1 (membrane form of HLA-G).

It emerges from these results that HLA-G is expressed by normal osteoblasts and pathological, in particular tumor, osteoblasts.

C) HLA-G is Expressed by the Normal Osteoblasts Generated in Culture from Mesenchymal Stem Cells

The expression of HLA-G by the osteoblasts generated in culture from mesenchymal stem cells was studied. Cultures of mesenchymal stem cells (MSCs) were incubated with various osteoblastic inducers, such as dexamethasone and Bone Morphogenetic Proteins (BMPs).

Expression of the mRNAs of alkaline phosphatase (ALP), of bone sialoprotein (BSP), of parathyroid hormone receptor 1 (PTHR1), of osteonectin (SPARC), of osterix transcription factor (osterix) and of α-smooth muscle actin (ASMA) was detected in the cultured cells of mesenchymal origin (see FIG. 4A). The combination of these various transcripts is known to be characteristic of osteoblasts (Cohen, 2006).

The resulting osteoblasts also expressed the HLA-G1, HLA-G2, HLA-G3, HLA-G4 and HLA-G5 mRNAs (see FIG. 4B).

The results obtained using the in situ immunofluorescence technique with the anti-HLA-G1/-G5 antibody (clone 87G) are represented in FIG. 5. The cells expressing the osteoblast markers were also positive for HLA-G1/-G5. However, it should be noted that the expression of HLA-G is significantly higher when the osteoblasts were induced with BMP-4 compared with the other BMPs (BMP-2 and BMP-7).

The results obtained using the flow cytometry technique are represented in FIG. 6. The majority of the osteoblasts express HLA-G1/-G5, as shown by the coexpression of HLA-G and of alkaline phosphatase (ALP). Nevertheless, this expression decreases over time: 66% of HLA-G+ osteoblasts were detected after 2 weeks of induction, whereas only 10% of HLA-G+ osteoblasts were detected after 4 weeks of induction.

Since MSCs can generate osteoblasts and chondroblasts and adipocytes, it was investigated whether HLA-G could be expressed by these other cell types. The soluble forms HLA-G5 and -G6 were detected by ELISA only in the supernatants of the osteogenic cultures (cells induced with BMP-4 and dexamethasone). Little or no soluble HLA-G was detected in the chondrogenic, adipogenic and angiogenic cultures (FIG. 7).

Moreover, the chondrocytes generated in culture by the micromass technique did not express HLA-G (FIG. 8). In addition, HLA-G was not detected in the adipocytes.

### EXAMPLE 2

### Demonstration of the Expression of HLA-G by Osteoblasts Derived from Osteosarcomas

1) Materials and methods

### Cells:

Osteosarcoma lines: HOS-154732 (McAllister et al., 1971), U2OS (Ponten et al., 1967), MG-63 (Billiau et al., 1977), SaOS2 (Fogh et al., 1975), CAL72 (Rocket et al., 1999) and SaOS2 RUNX2 DNN (Ghali et al., 2010).

The various biopsies studied came from the pathological anatomy department of the Trousseau Centre Hospitalier Universitaire (CHU) [university hospital teaching center] of Tours (France).

### Mesenchymal Stem Cells (MSCs):

The MSCs used came from healthy donors, treated in the department of orthopedic and trauma surgery of the CHU of Tours (France) and hospitalized for the implantation of a total hip replacement. The patient's informed consent was obtained in writing. 20 ml of bone marrow were taken from the posterior iliac crest during the procedure. These cells were used as a control for all the tests carried out in this study.

Cell cultures:
Standard culture: the cells of the various, lines were cultured in culture flasks (Falcon®, BD Biosciences, VWR, Strasbourg, France), at an initial density of 5000 cells per cm². The culture medium is composed of alpha-MEM Minimum Essential Medium (InVitrogen), with 10% (v/v) of fetal calf serum (FCS) (Perbio Hyclone, Logan, USA), 1% (v/v) L-glutamine, 1% (v/v) penicillin and streptomycin (InVitrogen) and 100 μM of fungizone (Bristol Myers Squibb, Rueil Malmaison, France). The culture medium was changed every 2 to 3 days.
The MSCs were cultured in an identical manner with the same culture medium, containing in addition FGF2 (AbCys, Paris, France) at 1 ng/ml.
Differentiation medium: for the osteocyte differentiation, the medium is composed of alpha MEM supplemented with 2% (v/v) FCS and 50 ng/ml of BMP4 (Peprotech, London, UK). The duration of this culture is from 8 to 10 days.

### Flow Cytometry:

200 000 cells were labeled using an antibody coupled to a fluorochrome: phycoerythrin (PE) or fluorescein isothiocyanate (FITC). To demonstrate the antigens expressed at the surface of the cells, the isolated and living cells were incubated for 45 minutes with the antibodies recognizing these antigens, washed, and then fixed with Cell Fix™ (Becton Dickinson, Erembodegem, France). To demonstrate the intracellular antigens, a prior step of cell permeabilization with CytoFix/CytoPerm™ (Becton Dickinson) is necessary before the immunolabeling. The negative control was obtained by means of a nonspecific immunoglobulin.

The cells were then run through the cytometer (FACS Calibur™, Becton Dickinson) with an Argon laser emitting at the wavelength of 488 nm. The result was interpreted using the CellQuest 3.1™ software.

The anti-HLA-G1, and -G5 antibodies 87G conjugated to alexa 488 and MEM/G9 conjugated to APC, or 4H84 (Exbio; Prague; Czech Republic), and an anti-ALPL antibody (R&D Systems) were used.

### Analyses by RT-PCR:

Trizol® Extraction:

After cell lysis using Trizol® (InVitrogen) and the addition of 200 μl of chloroform (Sigma) followed by centrifugation, three phases were obtained: the lower phase containing the proteins, the intermediate phase containing the deoxyribonucleic acids (DNAs) and the upper phase containing the ribonucleic acids (RNAs). Said upper phase was extracted and then precipitated from 500 μl of isopropanol (Sigma), then washed with 1 ml of 75% ethanol (Merck and Carbo Erba) and left to dry in ambient air until complete transparency was obtained. Finally, the RNA was dissolved in 50 ml of diethylpyrocarbonate (DEPC—for inhibiting RNAses) water.

RNA Assay:

Two microliters of RNA were diluted in 98 μl of DEPC water, and then placed in a quartz cuvette of the Gene Quant II dosimeter (Amersham Pharmacia, Sarclay, Orsay, France), after preparation of a blank (water alone). The assay was carried out by measuring the optical density by means of a laser emitting at 260 nm.

Reverse Transcription:

1 μg of RNA was diluted in DEPC water until 8 μl were obtained. The random hexamer and the dNTPs (Takara PrimeScript™ 1st strand cDNA synthesis kit (Takara Bio Inc.)) were added. After denaturation (5 min at 65° C.) in a thermocycler (Applied Biosystems, Foster City, Calif., USA), the Prime Script™ reverse transcriptase enzyme was added. This solution was incubated in the thermocycler, according to the following program: first step of 10 min at 30° C., second step of 60 min at 42° C., then third step of 5 min at 95° C. The complementary DNAs (cDNAs) obtained were stored at −20° C.

Polymerase Chain Reaction (PCR):

25 ng of cDNA were mixed with the dNTPs, with the primers targeting a sequence of interest (see table II hereinafter) and with the Taq polymerase enzyme (TaKara™ Ex Taq kit). The whole mixture was then incubated in the thermocycler, according to the following program composed of 35 cycles, each cycle consisting of 1 min at 98° C., followed by 30 seconds at 55° C., followed by 1 min at 72° C.

TABLE II

| | Primers: |
|---|---|
| | sense |
| Genes | antisense |
| GAPDH | ATCCCATCACCATCTTCCAGG |
| | (SEQ ID NO: 17) |
| | GAGGCAGGGATGATGTTCTGG |
| | (SEQ ID NO: 18) |
| ALPA | CTGGACCTCGTTGACACCTG |
| (or ALP) | (SEQ ID NO: 5) |
| | GACATTCTCTCGTTCACCGC |
| | (SEQ ID NO: 6) |
| Runx2 | GGCCCACAAATCTCAGATCGTT |
| | (SEQ ID NO: 19) |
| | CACTGGCGCTGCAACAAGAC |
| | (SEQ ID NO: 20) |
| Dlx5 | GCCACCAACCAGCCAGAGAA |
| | (SEQ ID NO: 21) |
| | GCGAGGTACTGAGTCTTCTGAAACC |
| | (SEQ ID NO: 22) |
| Osteonectin | ATCCCTGCCAGAACCACCACT |
| (SPARC) | (SEQ ID NO: 11) |
| | GCGCTTCTCATTCTCATGGATCT |
| | (SEQ ID NO: 12) |
| Col1a1 | ACATGGACCAGCAGACTGGCA |
| | (SEQ ID NO: 23) |
| | TCACTGTCTTGCCCCAGGCT |
| | (SEQ ID NO: 24) |

The PCR products were loaded onto a 1% agarose (Sigma) gel containing 0.01% of ethidium bromide (Sigma). The gel was then visualized using an ultraviolet lamp (Vilbert Lourmat, Eberhardzell, Germany). The bands were then analyzed using the Chemicapt™ software (BioRad, Calif., USA).

Quantitative Polymerase Chain Reaction (qPCR):

50 ng of cDNA were mixed with a solution containing a fluorescent molecule (Syber Green, InVitrogen). This solution was then placed in a well (96-well plate) in which the primers were deposited beforehand.

The plate was placed in the thermocycler (BioRad). The following program was used: 30 seconds at 95° C., 30 seconds at 56° C., 72° C. for 30 seconds, for 39 cycles. A melting curve was plotted in order to verify the quality of the DNAs amplified.

The amount of DNA was evaluated by means of the following formula:

2^{-(C(t) of the gene-C(t)GAPDH)} or 2^{-Δc(t)}

Western Blot:

The cells were detached, centrifuged, and then taken up in 500 μl of lysis buffer composed of 0.1% (v/v) Triton X-100 (Sigma). After centrifugation, the supernatant was removed and the protein extracts were then diluted in Laemmli buffer and then brought to boiling for 2 minutes.

The samples were assayed by measuring the optical density using the MRX II (Dynex technologies, Chantilly, USA), according to the Bradford technique.

The electrophoresis was carried out on a polyacrylamide gel with various concentrations depending on the protein of interest (10% for alkaline phosphatase and HLA-G); in a sodium dodecyl sulfate (SDS—Biorad) buffer. The proteins were blotted onto a polyvinylidene fluoride membrane, which was then saturated with milk proteins and incubated with the antibody of interest overnight. After application of the secondary antibody, visualization was carried out by chemiluminescence (ECL kit, Amersham).

Transfection:

The cells were transfected with 3 different siRNAs (InVitrogen) targeting RUNX2 (Select RNAi™, siRNA, catalog No. 1299003) or DLX5 (Stealth Select RNAi™, siRNA, catalog No. 1299003). Nonspecific siRNAs were used as controls (InVitrogen). 20 nM of siRNA were used for the transfections, which were carried out using the Amaxa Nucleofactor kit (Lonza, France), in accordance with the supplier's instructions. After 24 hours, the cells were induced to differentiate in the osteogenic differentiation medium. After 2, 4 and 6 days, the cells were harvested and tested for their expression of genes characteristic of osteoblasts or encoding HLA-G.

### 2) Results

The expression of the HLA-G1 (membrane HLA-G) and HLA-G5 (intracellular HLA-G) isoforms in various osteoblastic cell lines of human origin, CAL72, MG-63, HOS and U2OS, was studied by flow cytometry and by immunolabeling (Western blotting). The results are represented in FIG. 9. The HLA-G1 and HLA-G5 isoforms were detected in all the lines, but at varying degrees of expression. It was observed that HLA-G5 was always strongly expressed, whereas this was the case for HLA-G1 only in the CAL72 and MG-63 lines; HOS and U2OS expressing it significantly less.

The results of the investigation of HLA-G (HLA-G1 and HLA-G5) expression by immunohistochemistry in normal bone tissues (in the bone growth zone) and pathological bone tissues of human origin (in adults) are represented in table III below. It emerges from this table that only the normal or tumor osteoblasts and certain hypertrophic chondrocytes express HLA-G1 and -G5.

TABLE III
Expression of HLA-G1 and -G5 in normal or
pathological human bone tissues.

| Tissues | HLA-G expression |
|---|---|
| Normal tissues | |
| bone marrow endothelium | − |
| osteoclasts | − |
| trabecular osteoblasts | + |
| cortical osteoblasts | + |
| resting zone chondrocytes | − |
| proliferative zone chondrocytes | − |
| hypertrophic zone chondrocytes | ±^{a} |
| bone marrow adipocytes | − |
| vascular smooth muscle cells and | +^{b} |
| pericytes | |
| mesenchymal cells of the periosteum | + |
| mesenchymal cells of the | + |
| perichondrium | |
| Tumor tissues | |
| osteoblastic osteosarcomas | + |
| fibroblastic osteosarcomas | ± |
| osteoblastomas | + |
| chondrosarcomas | ± |
| chondroblastomas | − |
| Ewing's tumor cells | − |
| giant-cell tumors (GCTs) | − |
| normal osteoblasts induced by the | +^{c} |
| tumor | |

^{a}certain chondrocytes express HLA-G
^{b}HLA-G-positive perivascular cells in active bone formation sites
^{c}normal osteoblasts induced by bone tumors are all HLA-G-positive.

In order to confirm the specificity of HLA-G expression by osteoblasts, the expression of the DLX5 and RUNX2 genes involved in osteogenesis induction was inhibited by RNA interference. After transfection of bone marrow mesenchymal stem cells of human origin with siRNAs targeting DLX5 or RUNX2, the cells were cultured in an osteogenic medium (addition of BMP4). For 6 days, the expression of genes characteristic of osteoblasts (alkaline phosphatase or ALPL, osteonectin or SPARC, collagen 1α1 or COL1α1) and also HLA-G was studied. It was observed (see FIG. 10) that decreasing the expression of DLX5 and RUNX2 caused a decrease in the expression of the ALPL, SPARC, and COL1α1 genes, but also of HLA-G.

In order to confirm these results, 2 SaOS2 lines which express an inactivating form of RUNX2 (RUNX2 dominant negative or DNN) were used: the Δ6A2 and Δ4A5 lines.

The transduced line not containing RUNX2 DNN was used as a control line (C—). Δ4A5 strongly expresses DNN RUNX2, whereas its expression is more moderate in A6A2. The results are represented in FIG. 11.

Firstly, the expression of COL1α1 was studied by quantitative PCR (qRT-PCR) and Western blot since it is induced by RUNX2. A decrease in COL1α1 expression at the RNA level and at the protein level was then detected in the RUNX2 DNN lines. These decreases were significantly greater in the Δ4A5 line than in Δ6A2.

The expression of HLA-G (HLA-G1 and HLA-G5 isoforms) was then studied by flow cytometry and Western blot. The expression of HLA-G was found to be virtually extinguished in Δ4A5 and more weakly expressed in Δ6A2 than in the control line C—.

### LITERATURE

Betts M R et al., J Immunol. Methods, 281:65-78 (2003).
Billiau A et al., Antimicrob Agents Chemother., 12:11-15 (1977).
Carosella E D et al., Blood, 111:4862-4870 (2008a).
Carosella E D et al., Trends Immunol., 29:125-132 (2008b)
Cohen M M, Am J Med Genetics Part A, 140A:2646-2706 (2006).
Ellis S A et al., J. Immunol., 144:731-735 (1990).
Fogh J et al., New York: Plenum Press, 115-159 (1975).
Friedenstein A J et al., Exp Hematol., 4:267-274 (1976).
Geraghty D E et al., Proc Natl Acad. Sci. USA, 84:9145-9149 (1987).
Ghali O et al., Bone, 46:901-910 (2010).
Hackmon R et al., Fetal Diagn Ther., 19:404-409 (2004).
Ishitani A and Geraghty D E., Proc Natl Acad. Sci. USA, 89:3947-3951 (1992).
Kirszenbaum M et al., Proc Natl Acad. Sci. USA, 91:4209-4213 (1994).
Kirszenbaum M et al., Human Immunol., 43:237-241 (1995).
Le Discorde M et al. Biol Reprod., 73:280-288 (2005).
Le Rond S et al., Eur J Immunol., 34:649-660 (2004).
Le Rond S et al., J. Immunol., 176:3266-3276 (2006).
McAllister R M et al., Cancer, 27:397-402 (1971).
McMaster M et al., J. Immunol., 160:5922-5928 (1998).
Menier C et al., Hum Immunol., 64:315-26 (2003).

Moreau P et al., Human Immunol., 43:231-236 (1995).
Naji A, et al., Blood, 110:3936-3948 (2007).
Pittenger M F et al., Science, 284:143-147 (1999).
Ponten J et al., Int J Cancer, 2:434-447 (1967).
Rebmann V et al., Tissue Antigens, 53:14-22 (1999).
Rebmann V et al., Human Immunology, 66: 853-863 (2005).
Rochet N et al., Int J Cancer, 82:282-285 (1999).
Rouas-Freiss N et al., Cancer Res., 65:10139-10144 (2005).
Selmani Z et al., Stem Cells, 26:212-222 (2008).
Srivastava A, Medscape from Curr Med Res Opin., 21:1015-1026 (2005).
Ugurel S et al., Cancer, 92:369-376 (2001).
Vesper H, Medscape from Lab Med., 36:424-429 (2005).

1. An in vitro method for monitoring bone reconstruction in a subject, in whom a bone fracture has been diagnosed, the method comprising:
a) measuring a first concentration of at least one isoform of HLA-G in a first sample of biological fluid from the subject,
b) comparing the first concentration of the isoform of HLA-G measured in a) with a reference concentration of the isoform of HLA-G in a second sample of biological fluid from a healthy subject,
wherein the first concentration being higher than the reference concentration indicates bone reconstruction in the subject.

2. The method of claim 1, wherein the subject is a human being.

3. The method of claim 1, wherein the first and second samples of biological fluid are blood samples, and the first concentration and reference concentration are concentrations in plasma or serum.

4. The method of claim 3, wherein the measuring employs an immunological method.

5. The method of claim 1, wherein the at least one isoform of HLA-G is i) HLA-G1 and HLA-G5, or ii) HLA-G5 and HLA-G6.

6. The method of claim 5, wherein:
the subject is a human being,
the first and second samples of biological fluid are blood samples,
the measuring employs an immunological method, and
a plasma concentration of the HLA-G1 and HLA-G5 isoforms of greater than 20 ng/ml indicates bone reconstruction in the subject.

7. The method of claim 5, wherein:
the subject is a human being,
the first and second samples of biological fluid are blood samples,
the measuring employs an immunological method, and
a plasma concentration of the HLA-G5 and HLA-G6 isoforms of greater than 10 ng/ml indicates bone reconstruction in the subject.

8. The method of claim 5, wherein:
the subject is a human being,
the first and second samples of biological fluid are blood samples,
the measuring employs an immunological method, and
a serum concentration of the HLA-G1 and HLA-G5 isoforms of greater than 25 ng/ml indicates bone reconstruction in the subject.

9. An in vitro method for monitoring a change in a bone tumor in a subject, using biological samples from the subject obtained at a time t0 and at a time t1, the method comprising a) determining a concentration or b) quantitatively determining an expression of at least one isoform of HLA-G in the biological samples,
wherein an increase in the concentration or in the expression level of the isoform of HLA-G between the times t0 and t1 indicates a progression of the bone tumor in the subject, and
wherein a decrease in the concentration or in the expression level of the isoform of HLA-G between the times t0 and t1 indicates a remission of said the bone tumor in the subject.

10. The method of claim 9, wherein the subject is a human being.

11. The method of claim 9, wherein the biological samples are blood samples, and the method comprises determining the concentration of the isoform of HLA-G in plasma or serum.

12. The method of claim 9, wherein the biological samples comprise osteoblasts obtained by bone biopsy of the tumor, and the method comprises quantitatively determining the expression of the isoform of HLA-G by the osteoblasts.

13. The method of an appropriate of claim 9, wherein the a) determining a concentration, or the b) quantitatively determining an expression, employs an immunological method.

14. The method of claim 12, wherein the expression, by the osteoblasts, of the isoform of HLA-G is quantitatively determined by detecting an mRNA encoding the isoform of HLA-G.

15. The method of claim 9, wherein the bone tumor is selected from the group consisting of an osteosarcoma, an osteoblastoma, a Ewing's sarcoma and a giant-cell tumor.

16. The method of claim 9, wherein the at least one isoform of HLA-G is i) HLA-G1 and HLA-G5, or ii) HLA-G5 and HLA-G6.

17. An in vitro method of screening for an agent which modulates osteogenesis, the method comprising:
a) quantitatively determining a first expression of at least one isoform of HLA-G by osteoblasts;
b) contacting the osteoblasts with a test agent; then
c) quantitatively determining a second expression of the isoform of HLA-G by the osteoblasts,
wherein a difference in a level of the first and second expressions indicates that the agent modulates osteogenesis.

18. The method of claim 17, wherein the at least one isoform of HLA-G is i) HLA-G5, or ii) HLA-G5 and HLA-G6.

19. An isoform of HLA-G or a nucleic acid molecule encoding an isoform of HLA-G, isolated from a subject, suitable for use as an osteogenesis marker in the subject.

20. The method of claim 4, wherein the immunological method is ELISA.
